(19) United States
(12) Patent Application Publication
(10) Pub. No.: US 2007/0059742 A1
(43) Pub. Date: Mar. 15, 2007

(54) Process of stripping a microarray for reuse

(76) Inventors: Axel G. Stover, Shoreline, WA (US); Andy McShea, Kingston, WA (US); Michael J. Lodes, Seattle, WA (US); Kristian M. Roth, Bothell, WA (US); Kevin Robert Schwarzkopf, Seattle, WA (US)

Correspondence Address: COMBIMATRIX CORPORATION, 6500 HARBOUR HEIGHTS PARKWAY, MUKILTEO, WA, 98275, US

(21) Appl. No.: 11/499,230

(22) Filed: Aug. 3, 2006

Related U.S. Application Data
(60) Provisional application No. 60/715,847, filed on Sep. 9, 2005.

(51) Int. Cl. C12Q 1/68; C12M 1/34; H01L 21/00

(52) U.S. Cl. 435/6; 435/287.2; 438/1; 257/E51.02

(57) ABSTRACT

Disclosed herein is a process for stripping oligonucleotide target from a microarray to allow reuse of the microarray. The process comprises providing a microarray having probe oligonucleotides attached thereto and target oligonucleotides hybridized to the probe oligonucleotides. The microarray is then incubated with a formulation comprising an organic solvent and an organic base. The target oligonucleotides are substantially removed from the microarray by the formulation. Alternatively, prior to or after incubation of the microarray with the formulation, the microarray may be contacted to an aqueous solution of a base to improve the efficiency of removal of the target oligonucleotides.

### CROSS REFERENCE TO RELATED APPLICATIONS

This nonprovisional application claims the benefit of provisional application Ser. No. 60/715,847, filed 9 Sep. 2005, under 35 U.S.C §119(e).

### TECHNICAL FIELD

Disclosed herein is a process of stripping a microarray for reuse. More specifically, disclosed herein is a process for stripping hybridized target oligonucleotides from a microarray while substantially leaving intact probe oligonucleotides on the microarray. After stripping, the microarray can be reused for further experiments.

### BACKGROUND

Microarray preparation methods include the following: (1) spotting a solution on a prepared flat surface using spotting robots; (2) in situ synthesis by printing reagents via ink jet or other printing technology and using regular phosphoramidite chemistry; (3) in situ parallel synthesis using electrochemically-generated acid for deprotection and using regular phosphoramidite chemistry; (4) maskless photo-generated acid (PGA) controlled in situ synthesis and using regular phosphoramidite chemistry; (5) mask-directed in situ parallel synthesis using photo-cleavage of photolabile protecting groups (PLPG); (6) maskless in situ parallel synthesis using PLPG and digital photolithography; and (7) electric field attraction/repulsion for depositing oligonucleotides. A review of oligonucleotide microarray synthesis is provided by: Gao et al., Biopolymers 73:579, 2004.

Photolithographic techniques for in situ oligonucleotide synthesis are disclosed in Fodor et al. U.S. Pat. No. 5,445,934 and the additional patents claiming priority thereto and Pirrung et al. U.S. Pat. No. 5,405,783, the disclosure of each is incorporated by reference herein. Electric field attraction/repulsion microarrays are disclosed in Hollis et al. U.S. Pat. No. 5,653,939, the disclosure of which is incorporated by reference herein, and Heller et al. U.S. Pat. No. 5,929,208, the disclosure of which is incorporated by reference herein. Pin printing techniques (spotting) for mechanical deposition of macromolecules is disclosed in Martinsky U.S. Pat. No. 6,101,946, the disclosure of which is incorporated by reference herein. Spotting by means of micropipettes is disclosed in Gordon, et al. U.S. Pat. No. 5,601,980, the disclosure of which is incorporated by reference herein. Spotting by means of ink jet printing is disclosed in Papen, et al. U.S. Pat. No. 5,927,547, the disclosure of which is incorporated by reference herein. An electrode microarray for in situ oligonucleotide synthesis using electrochemical deblocking is disclosed in Montgomery, U.S. Pat. Nos. 6,093,302, 6,280,595, and 6,444,111 (Montgomery I, II, and III respectively), the disclosure of each is incorporated by reference herein. A review of oligo microarray synthesis is provided by: Gao et al., Biopolymers 2004, 73:579.

Microarray substrates may be composed of glass slides, complementary metal oxide semiconductor (CMOS) materials, or membranes. These substrates may have a coating material adhered to the surface or may have a linker covalently attached to the surface. Oligonucleotides are attached to the coating or linker. Typically, single stranded DNA or other oligonucleotides (probe materials) are attached to a microarray or synthesized in situ on a microarray at defined locations. Printed or spotted cDNA microarrays typically used double stranded DNA.

As advised by the manufacturers, such microarrays are often used only one time for a hybridization experiment and then discarded afterwards. However, as a cost-savings means, Researchers often will try to dehybridize target material from the target probes on a microarray using a high stringency technique in order to reuse the microarray. Common high stringency techniques generally include a combination of various salts, solvents, and relatively high temperatures. However, even though such approaches often succeed in melting off hybridized target nucleic acids from the attached oligonucleotide probes, the probes or the microarray surface may be damaged or even removed during the process. Thus, the microarray may become incapable of reuse. The problem of probe damage or removal from attempted reuse is often found for microarrays made by photolithography processes where the attached oligonucleotide probes are easily damaged. Typically, the high stringency approaches provide a limited ability to reuse a microarray for one or possibly two or more hybridization experiments.

High stringency techniques to remove hybridized material include decreasing salt concentration by using pure water to wash the microarray, adding surfactants, increasing the temperature of solution in contact with the microarray, or a combination of the aforementioned. Most often, the solutions and conditions chosen reflect the type of microarray and the type of coating on the microarray. However, these methods can lead to incomplete removal, and in the case of using high temperature, these methods can damage the surface of a microarray that anchors the probe materials. The process disclosed herein addresses the problems related to the number of times of reuse, complete removal of target material, and minimizing damage to the microarray during reuse treatment.

### SUMMARY

Disclosed herein is a process for stripping a microarray for reuse. In one embodiment, the process comprises providing a microarray having probe oligonucleotides attached thereto and target oligonucleotides hybridized to the probe oligonucleotides; and incubating the microarray with a formulation comprising an organic solvent and an organic base. The formulation substantially removes the target oligonucleotides from the microarray. Preferably, the microarray is formed by spotting or in situ synthesis. In another embodiment, the step of incubating the microarray with a formulation comprising an organic solvent and an organic base, further comprises: contacting the microarray to an aqueous base solution. The additional step further removes target oligonucleotide.

Further disclosed herein is another embodiment for a process for stripping a microarray for reuse. The process comprises: providing a microarray having probe oligonucleotides attached thereto and target oligonucleotides hybridized to the probe oligonucleotides; contacting the microarray to an aqueous base solution; and incubating the microarray with a formulation comprising an organic solvent and an organic base. The combination of the aqueous base solution and the formulation substantially remove the target oligonucleotides from the microarray.

### BRIEF DESCRIPTION OF THE DRAWINGS

FIG. 1 provides images of a microarray before and after stripping of target RNA using a formulation comprising about 50% ethanolamine and about 50% ethanol (by volume) at about 65° C. for a time of about one hour.

FIG. 2 provides images of a microarray before and after stripping of target RNA using a formulation comprising about 50% ethanolamine and about 50% ethanol (by volume) at about 65° C. for a time of about one hour.

FIG. 3 provides an image of a microarray after stripping of target RNA using a formulation comprising about 50% ethanolamine and about 50% ethanol (by volume) at about 65° C. for a time of about one hour. The image shows the microarray at a higher magnification than the images in FIG. 2.

FIG. 4 provides an image of a microarray after stripping of target RNA using a solution comprising hot water at about 65° C. for a time of about one hour. Incomplete removal of the target RNA is shown.

FIG. 5 provides images of a microarray before and after stripping of target RNA using a solution of 50 millimolar potassium carbonate in water at 65° C. for a time of one hour.

FIG. 6 provides an image of a microarray after stripping of target RNA using a solution of 50 millimolar potassium carbonate in water at 65° C. for a time of one hour. The image shows the microarray at a higher magnification than the images in FIG. 5. Incomplete removal of the target RNA is shown.

FIG. 7 provides images of a microarray before and after stripping of target RNA using a solution of concentrated ammonium hydroxide at 65° C. for a time of one hour.

FIG. 8 provides images of a microarray before and after stripping of target RNA using a formulation of about 50% ethanolamine and about 50% ethanol (by volume) at room temperature and at 65° C., both for a time of one hour.

FIG. 9 provides images of a microarray before and after stripping of target RNA using a formulation of about 50% ethanolamine and about 50% ethanol (by volume) at 65° C. for a time of 15 minutes compared to one hour.

FIG. 10 provides images of three different microarrays subjected to three different types of stripping solutions for reuse of the microarrays.

### DETAILED DESCRIPTION

Disclosed herein is a process for stripping a microarray for reuse. In one embodiment, the process comprises providing a microarray having probe oligonucleotides attached thereto and target oligonucleotides hybridized to the probe oligonucleotides; and incubating the microarray with a formulation comprising an organic solvent and an organic base. The oligonucleotides may be DNA or RNA or a combination thereof. The formulation substantially removes the target oligonucleotides from the microarray. Preferably, the microarray is formed by spotting or in situ synthesis. More preferably, the microarray is an electrode-containing microarray, wherein the probe oligonucleotides are attached to a porous reaction layer covering the electrodes of the electrode-containing microarray. Preferably, the porous reaction layer is sucrose but could be any one of the materials or a mixture of the materials disclosed in U.S. patent application Ser. No. 10/992,252, filed 18 Nov. 2004, the disclosure of which is incorporated by reference herein. Preferably, the electrodes are platinum.

Preferably, the temperature of incubating is from about room temperature to about 75 degrees Celsius. Preferably, the time of incubating is from about 1 minute to about 24 hours. More preferably, the time of incubating is about one hour and the temperature of incubating is about 65 degrees Celsius.

Preferably, the organic solvent concentration is about 1 to 99 percent by volume. More preferably, the organic solvent concentration is about 50 percent by volume. Preferably, the organic solvent is ethanol. Alternatively, the organic solvent is one of or a combination of the following solvents: ethanol, isopropanol, 1,1,1-trichloroethane, 1,1,2-trichloro-1,2,2-trifluoroethane, 1,1,2-trichloroethane, 1,4-dichlorobenzene, 1-butanol, 2-butanol, isobutanol, tert-butanol, 1-hexene, 1-propanol, 2-(2-butoxyethoxy)ethyl acetate, 2-butoxyethanol acetate, 2-butoxyethyl acetate, 2-ethoxyethanol acetate, 2-ethoxyethanol, 2-methoxyethanol acetate, 2-methoxyethanol, 2-methylhexane, 2-nitropropane, acetic acid, acetone alcohol, acetone, acetonitrile, allyl alcohol, benzene, benzotrifluoride, benzyl chloride, biphenyl, carbon disulfide, carbon tetrachloride, chlorobenzene, chlorobromomethane, cyclodecane, cycloheptane, cyclohexane, cyclohexanol, cyclohexanone, cyclononane, cyclooctane, cyclopentane, diacetone alcohol, dibromomethane, dichlorodiphenyltrichloroethane, dichloroethene, diemthyl sulfoxide, diethanolamine, diethyl ether, diethylene glycol, dimethyl ethanolamine, dimethyl formamide, dipropylene glycol, ethanol, ethyl acetate, ethyl benzene, ethyl ether, ethyl glycol acetate, ethyl glycol, ethylbenzene, ethylene glycol, formamide, formic acid, furfural, furfuryl alcohol, heptafluorocyclopentane, heptafluoropropyl methyl ether, heptane, hexachlorocyclohexane, hexane, isoamyl alcohol, isobutyl acetate, isobutyl alcohol, isobutyl isobutyrate, isomethoxynonafluorobutane, iso-methoxynonafluorobutane, isophorone, isopropyl acetate, iso-propyl alcohol, isopropylamine-striazine, methanol, methoxy propyl acetate, methyl amyl ketone, methyl chloride, methyl chloroform, methyl ethyl ketone, methyl glycol acetate methyl isobutyl ketone, methyl propyl ketone, methylene chloride, monochlorotoluene, monothiophosphate, n-amyl alcohol, n-butyl acetate, n-butyl alcohol, n-decane, nitrobenzene, nitromethane, n-methoxynonafluorobutane, n-methylpyrrolidone, n-nonane, n-octane, n-octyl alcohol, n-butyl acetate, n-methoxynonafluorobutane, n-pentane, n-propyl acetate, n-propyl alcohol, ortho-dichlorobenzene, perchloroethene, perchloroethylene, propylene glycol diacetate, propylene glycol, pyridine, t-amyl alcohol, t-butyl alcohol, tetrachloroethylene, tetrahydrofuran, toluene, trans-1,2-dichloroethylene, trichloroethene, trichloroethylene, trichlorofluoromethane, triethanolamine, triethylene gycol, vinyl choloride, and xylene.

Preferably, the organic base is selected from the group consisting of ethanolamine and ethylenediamine and combinations thereof. Alternatively, the organic base is one of or a combination of the following: ethanolamine, ethylenediamine, adenine, guanine, cytocine, thymine, uracil, methylamine, ethyleneimine, dimethylamine, ethylamine, cysteamine, 1,2-ethanediamine, azetidine, propylamine, trimethylamine, 1-amino-2-methoxoythane, 1,2-propanediamine, 1,3-propanediamine, 1,2,3 triaminopropane, allantoin, pyrrolidine, morpholine, N,N-dimethylglycine, piperazine, butylamine, sec-butylamine, tert-butylamine, diethylamine, 1,4-butanediamine, 1,2 dimethylaminoethane, 4-pyridinamine, N-methylpyrrolidine, piperidine, 1 amino 2,2 dimethylpropane, diethylmethylamine, 3-methyl-1-butanamine, 2 methyl 1 butanamine, 3-pentanamine, pentylamine, cadaverine, cyclohexylamine, 1,2 dimethylpyrrolidine, 1-methylpiperidine, 3-amino-3-methylpentane, diisopropylamine, hexylamine, triethylamine, hexamethylenediamine, benzylamine, 1,2-dimethylpiperidine, 1 ethylpiperidine, 2-heptanamine, heptylamine, 2,2,4-trimethylpiperidine, dibutylamine, N methyl-2-heptanamine, octylamine, 1-butylpiperidine, 2,2,6,6-tetramethylpiperidine, nonylamine, tryptamine, ephedrine, bornylamine, neobornylamine, butylcyclohexylamine, decylamine, undecylamine, dodecylamine, tridecylamine, tetradecylamine, pentadecyalamine, hexadecylamine, and combinations thereof.

In another embodiment, the step of incubating the microarray with a formulation comprising an organic solvent and an organic base, further comprises: contacting the microarray to an aqueous base solution. The additional step further removes target oligonucleotide. Preferably, the aqueous base solution is at a concentration of about 0.01 molar to about 5 molar. Preferably, the aqueous base solution has a base selected from the group consisting of sodium hydroxide, potassium hydroxide, and ammonium hydroxide and combinations thereof.

Preferably, the temperature of contacting the microarray to the organic base is about 2 to 95 degrees Celsius. Preferably, the time of contacting is about 1 minute to about 60 minutes. More preferably, the temperature of contacting is about 20 degrees Celsius and the time of contacting is about 15 minutes. More preferably, the aqueous base solution is sodium hydroxide at a concentration of about 0.5 molar.

Further disclosed herein is another embodiment for a process for stripping a microarray for reuse. The process comprises: providing a microarray having probe oligonucleotides attached thereto and target oligonucleotides hybridized to the probe oligonucleotides; contacting the microarray to an aqueous base solution; and incubating the microarray with a formulation comprising an organic solvent and an organic base. The combination of the aqueous base solution and the formulation substantially remove the target oligonucleotides from the microarray. The oligonucleotides may be DNA or RNA or a combination thereof.

Preferably, the microarray is formed by spotting or in situ synthesis. More preferably, the microarray is an electrode-containing microarray, wherein the probe oligonucleotides are attached to a porous reaction layer covering the electrodes of the electrode-containing microarray. Preferably, the porous reaction layer is sucrose. Preferably, the electrodes are platinum.

Preferably, the aqueous base solution is at a concentration of about 0.01 molar to about 5 molar. Preferably, the aqueous base solution has a base selected from the group consisting of sodium hydroxide, potassium hydroxide, and ammonium hydroxide and combinations thereof.

Preferably, the temperature of contacting is about 2 to 95 degrees Celsius. Preferably, the time of contacting is about 1 minute to about 60 minutes. More preferably, the temperature of contacting is about 20 degrees Celsius and the time of contacting is about 15 minutes. More preferably, the aqueous base solution is sodium hydroxide at a concentration of about 0.5 molar.

Preferably, the temperature of incubating is from about room temperature to about 75 degrees Celsius. Preferably, the time of incubating is from about 1 minute to about 24 hours. More preferably, the time of incubating is about one hour and the temperature of incubating is about 65 degrees Celsius.

Preferably, the organic solvent concentration is about 1 to 99 percent by volume. More preferably, the organic solvent concentration is about 50 percent by volume. Preferably, the organic solvent is ethanol. Alternatively, the organic solvent is one of or a combination of the following: ethanol, isopropanol, 1,1,1-trichloroethane, 1,1,2-trichloro-1,2,2-trifluoroethane, 1,1,2-trichloroethane, 1,4-dichlorobenzene, 1-butanol, 2-butanol, isobutanol, tert-butanol, 1-hexene, 1 propanol, 2-(2-butoxyethoxy)ethyl acetate, 2-butoxyethanol acetate, 2-butoxyethyl acetate, 2 ethoxyethanol acetate, 2-ethoxyethanol, 2-methoxyethanol acetate, 2-methoxyethanol, 2 methylhexane, 2-nitropropane, acetic acid, acetone alcohol, acetone, acetonitrile, allyl alcohol, benzene, benzotrifluoride, benzyl chloride, biphenyl, carbon disulfide, carbon tetrachloride, chlorobenzene, chlorobromomethane, cyclodecane, cycloheptane, cyclohexane, cyclohexanol, cyclohexanone, cyclononane, cyclooctane, cyclopentane, diacetone alcohol, dibromomethane, dichlorodiphenyltrichloroethane, dichloroethene, diemthyl sulfoxide, diethanolamine, diethyl ether, diethylene glycol, dimethyl ethanolamine, dimethyl formamide, dipropylene glycol, ethanol, ethyl acetate, ethyl benzene, ethyl ether, ethyl glycol acetate, ethyl glycol, ethylbenzene, ethylene glycol, formamide, formic acid, furfural, furfuryl alcohol, heptafluorocyclopentane, heptafluoropropyl methyl ether, heptane, hexachlorocyclohexane, hexane, isoamyl alcohol, isobutyl acetate, isobutyl alcohol, isobutyl isobutyrate, isomethoxynonafluorobutane, iso-methoxynonafluorobutane, isophorone, isopropyl acetate, iso-propyl alcohol, isopropylamine-striazine, methanol, methoxy propyl acetate, methyl amyl ketone, methyl chloride, methyl chloroform, methyl ethyl ketone, methyl glycol acetate methyl isobutyl ketone, methyl propyl ketone, methylene chloride, monochlorotoluene, monothiophosphate, n-amyl alcohol, n-butyl acetate, n-butyl alcohol, n-decane, nitrobenzene, nitromethane, n-methoxynonafluorobutane, n methylpyrrolidone, n-nonane, n-octane, n-octyl alcohol, n-butyl acetate, n methoxynonafluorobutane, n-pentane, n-propyl acetate, n-propyl alcohol, ortho-dichlorobenzene, perchloroethene, perchloroethylene, propylene glycol diacetate, propylene glycol, pyridine, t-amyl alcohol, t-butyl alcohol, tetrachloroethylene, tetrahydrofuran, toluene, trans-1,2-dichloroethylene, trichloroethene, trichloroethylene, trichlorofluoromethane, triethanolamine, triethylene gycol, vinyl choloride, xylene, and combinations thereof.

Preferably, the organic base is one of or a combination of the following: ethanolamine and ethylenediamine. Preferably, the organic base is one of or a combination of the following: adenine, guanine, cytocine, thymine, uracil, methylamine, ethyleneimine, dimethylamine, ethylamine, cysteamine, 1,2-ethanediamine, azetidine, propylamine, trimethylamine, 1-amino-2-methoxoythane, 1,2-propanediamine, 1,3-propanediamine, 1,2,3 triaminopropane, allantoin, pyrrolidine, morpholine, N,N-dimethylglycine, piperazine, butylamine, sec-butylamine, tert-butylamine, diethylamine, 1,4-butanediamine, 1,2 dimethylaminoethane, 4-pyridinamine, N-methylpyrrolidine, piperidine, 1 amino 2,2 dimethylpropane, diethylmethylamine, 3-methyl-1-butanamine, 2 methyl 1 butanamine, 3-pentanamine, pentylamine, cadaverine, cyclohexylamine, 1,2 dimethylpyrrolidine, 1-methylpiperidine, 3-amino-3-methylpentane, diisopropylamine, hexylamine, triethylamine, hexamethylenediamine, benzylamine, 1,2-dimethylpiperidine, 1 ethylpiperidine, 2-heptanamine, heptylamine, 2,2,4-trimethylpiperidine, dibutylamine, N methyl-2-heptanamine, octylamine, 1-butylpiperidine, 2,2,6,6-tetramethylpiperidine, nonylamine, tryptamine, ephedrine, bornylamine, neobornylamine, butylcyclohexylamine, decylamine, undecylamine, dodecylamine, tridecylamine, tetradecylamine, pentadecyalamine, hexadecylamine, octadecylamine and combinations thereof.

### EXAMPLE 1

### Ethanolamine and Ethanol Stripping Solution

In this example, a CombiMatrix CustomArray™ 12k microarray was used to synthesize oligonucleotides attached to the microarray. The microarray had approximately 12,000 platinum surfaced electrodes on a solid surface having a porous reaction layer over the Pt electrode surface, wherein each electrode was electronically addressable via computer control. The probe oligonucleotides were single-stranded DNA and were synthesized in situ using electrochemical synthesis at locations associated with the electrodes on the microarray. The electrochemical synthesis used standard phosphoramidite chemistry coupled with electrochemical deblocking of the protecting groups on the synthesized DNA for the addition of each nucleotide contained in the oligonucleotide.

The microarray had a porous reaction layer having organic reactive hydroxyl groups that allowed attachment of the first phosphoramidite base. The porous reaction layer was sucrose. Each electrode site intended for deblocking had the electrode turned on (i.e., current applied) to electrochemically generate acid sufficient to remove the acid-labile protecting group. Buffer in the solution was used to confine the acidic environment to the activated electrode site and not to neighboring electrodes. Removal of the protecting group allowed addition of the next phosphoramidite. Oligonucleotide probe DNA synthesized on the microarray was and average of 35 nucleotides in length and was designed to be complementary to portions of immunological genes for cytokines and chemokines.

The target RNA samples came from HEK-293 cells and were labeled with biotin-11-CTP and biotin-16-UTP. The blocking and labeling procedure for the biotinylated samples was as follows: Wash solution was removed from a hybridization chamber that covered the microarray active surface. Blocking solution was then added to the chamber. The blocking solution comprised two times phosphate buffered saline (2×PBS), 0.1% TWEEN® 20, and 1% bovine serum albumin (BSA). The microarray having the blocking solution was incubated for 15 minutes at room temperature. The blocking solution was removed. A labeling solution was added to the chamber, and the microarray was incubated for 30 minutes at room temperature while protected from light. The labeling solution comprised streptavidin-conjugated fluorochrome (streptavidin-Cy5® diluted at 1:1000), 2×PBS, 0.1% TWEEN® 20, 1% BSA. Hybridization was conducted at 45° C. for 16 hours.

The Cy5®-labeled RNA sample was hybridized to the microarray at 45° C. for 16 hours. The hybridization solution comprised four micrograms of labeled target RNA, 2×PBS, and 0.1% TWEEN® 20. After hybridization, the microarray was washed to remove excess unbound labeled RNA. The microarray was then scanned using an Axon scanner. The microarray was then exposed to a stripping solution comprising equal parts by volume of absolute ethanol (200 proof) and ethanolamine (>99.5%). The temperature was about 65° C. and the time was about one hour.

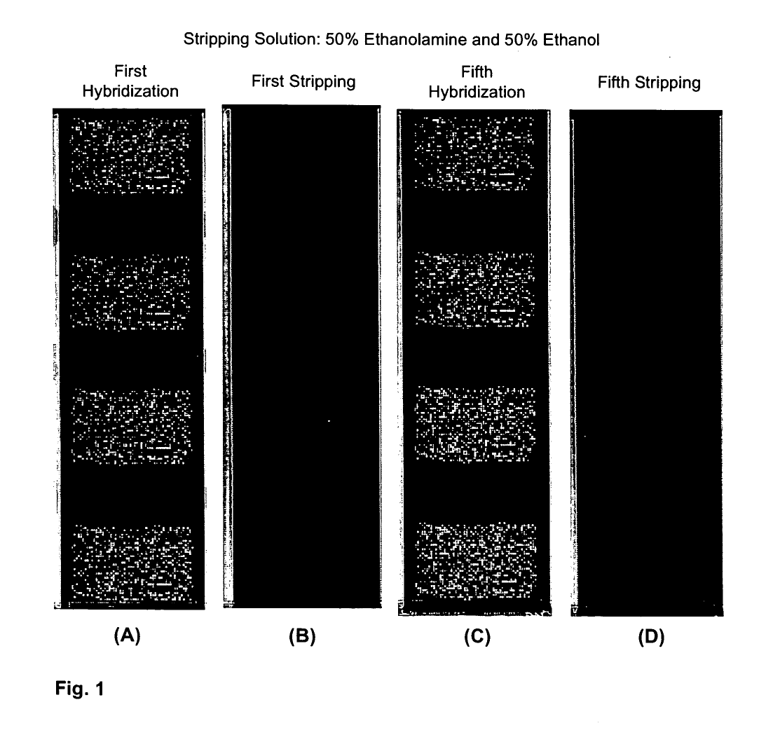

FIG. 1 shows images of portions of a microarray after hybridization and stripping. The stripping solution was 50% ethanolamine and 50% ethanol by volume. Altogether, five hybridization and stripping sequences were performed on the same microarray. A scanned image was taken after each hybridization step. The first hybridization and stripping sequence and the last (fifth) hybridization and stripping sequence are shown. FIG. 1 demonstrates that very little fluorescence was lost after five sequences of hybridization stripping of the labeled RNA from the micro array.

### EXAMPLE 2

### Ethylenediamine and Ethanol Stripping Solution

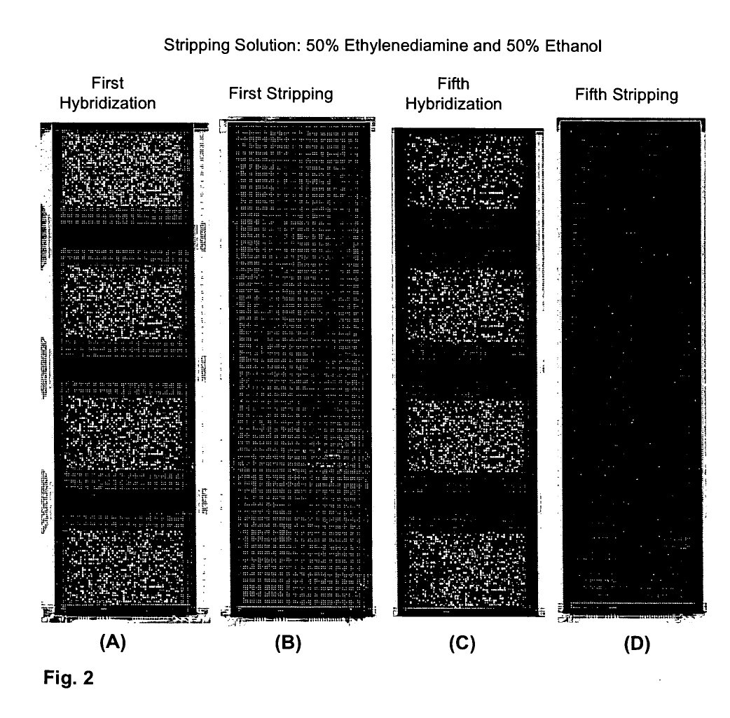

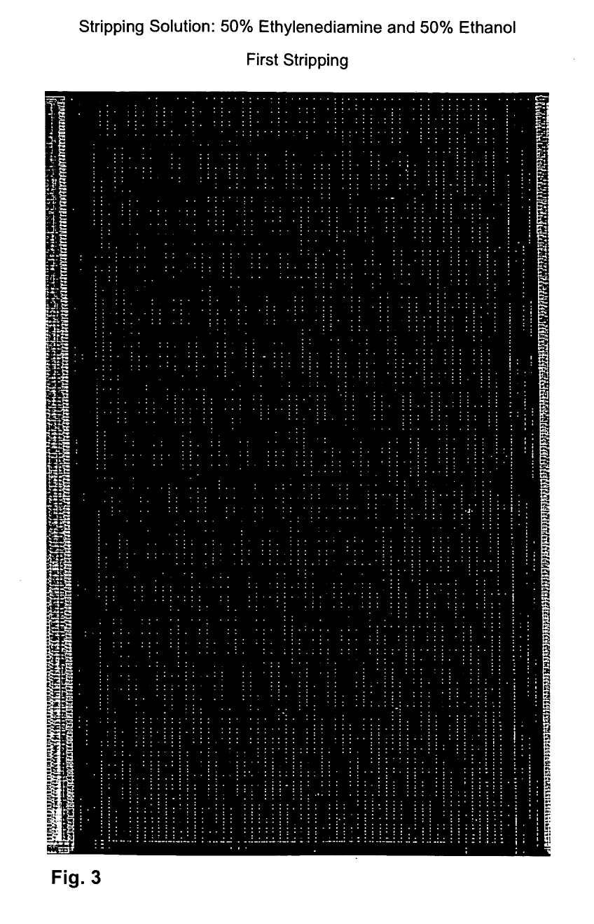

A microarray was prepared according to the procedures of Example 1. The stripping solution for removal of the labeled RNA comprised (by volume) 50% ethylenediamine and 50% ethanol (200 proof). The results were essentially the same as in Example 1. FIG. 2 shows images of the microarray before and after stripping. FIG. 3 shows a higher magnification image of the microarray after stripping. As can be seen in the figures, the stripping solution removed the labeled RNA and subsequent hybridization to the stripped microarray was successful.

### EXAMPLE 3

### Hot Water

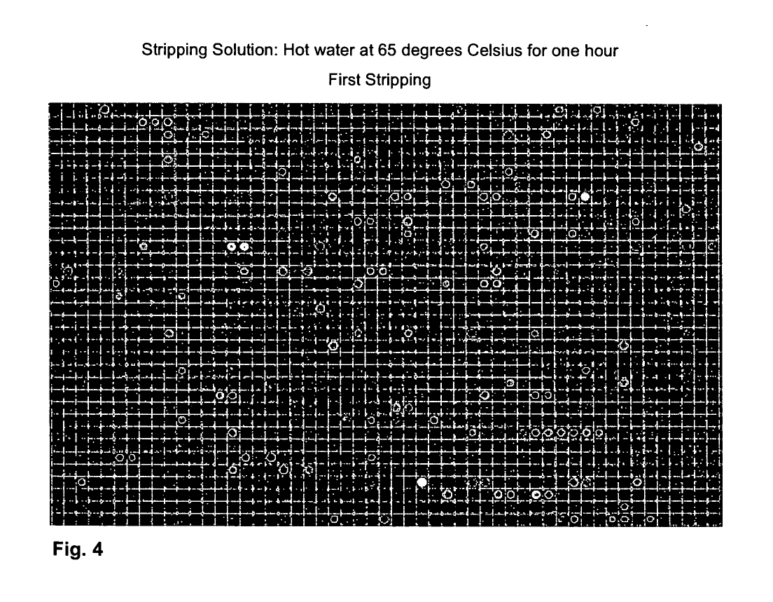

A microarray was prepared according to the procedures of Example 1. The stripping solution for removal of the labeled RNA comprised hot water. The temperature of the water was about 65° C. and the time of exposure was about one hour. Although hot water removed the majority of hybridized material, enough remained behind to cause problems with further use of the microarray. FIG. 4 provides an image of the microarray after stripping of target RNA. Incomplete removal of the target RNA is shown as evidence by residual fluorescence on the microarray.

### EXAMPLE 4

### Potassium Carbonate

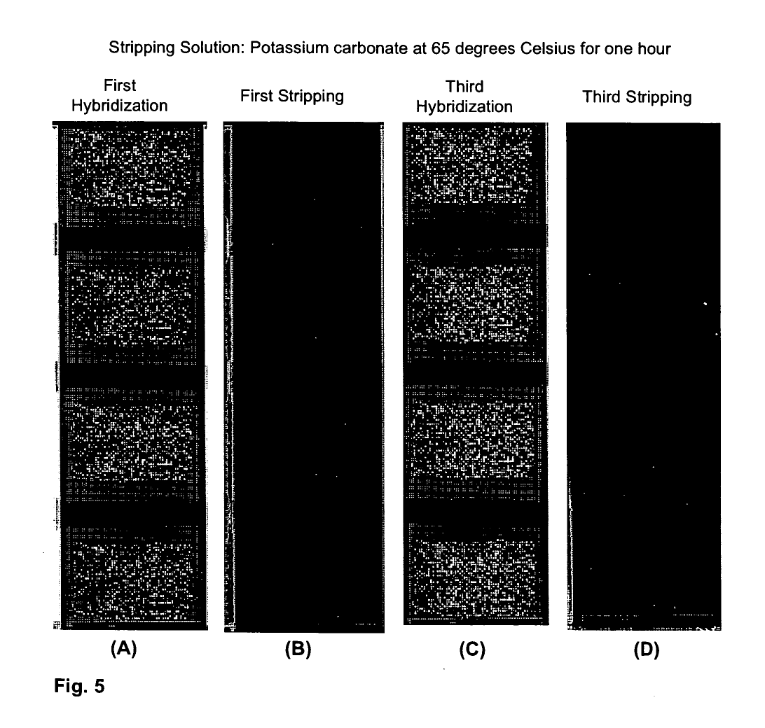

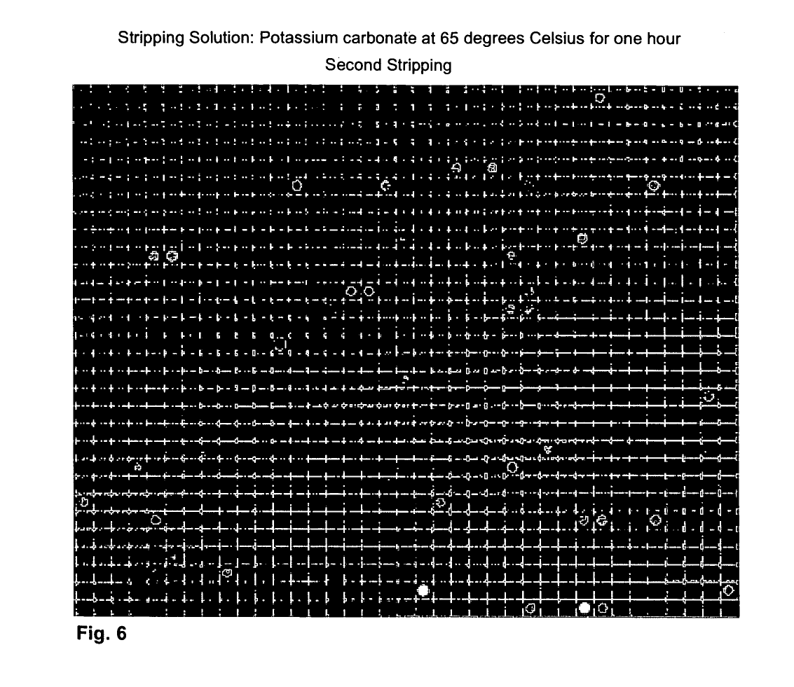

A microarray was prepared according to the procedures of Example 1. The stripping solution for removal of the labeled RNA comprised potassium carbonate in water at a concentration of 50 millimolar. The stripping conditions were about 1 hour at about 65° C. FIG. 5 shows images of the microarray before and after stripping. FIG. 6 shows a higher magnification image of the microarray and shows that there was incomplete removal of the hybridized labeled-RNA as evidenced by the residual fluorescence.

### EXAMPLE 5

### Ammonium Hydroxide

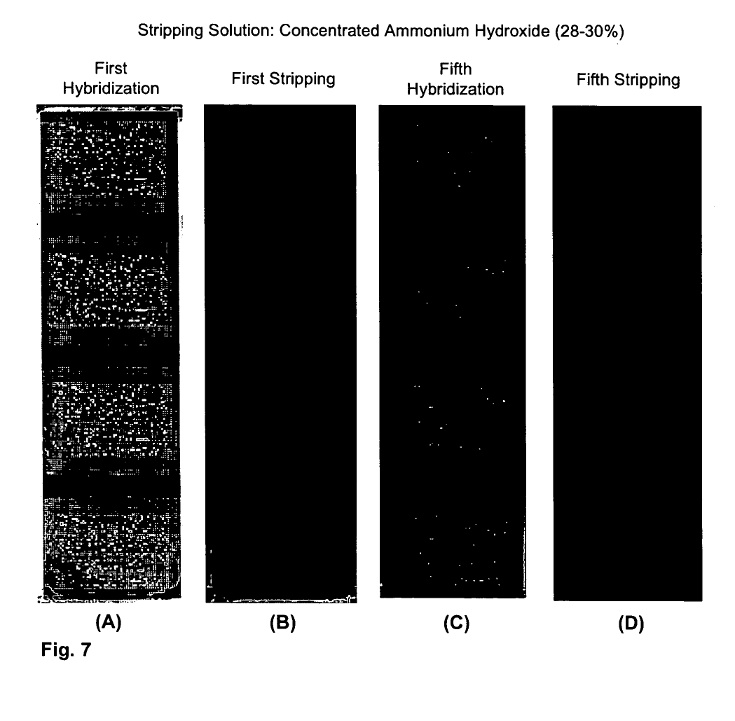

A microarray was prepared according to the procedures of Example 1. The stripping solution for removal of the labeled RNA comprised concentrated ammonium hydroxide as sold by the vendor (28-30% in water). Ammonium hydroxide stripped the hybridized material but appeared to have damaged the microarray during the first stripping sequence. The results are shown in FIG. 7. After the first stripping, subsequent hybridization produced only faint spots thus indicating damage to the microarray.

### EXAMPLE 6

### Ethanolamine and Ethanol Stripping Temperature

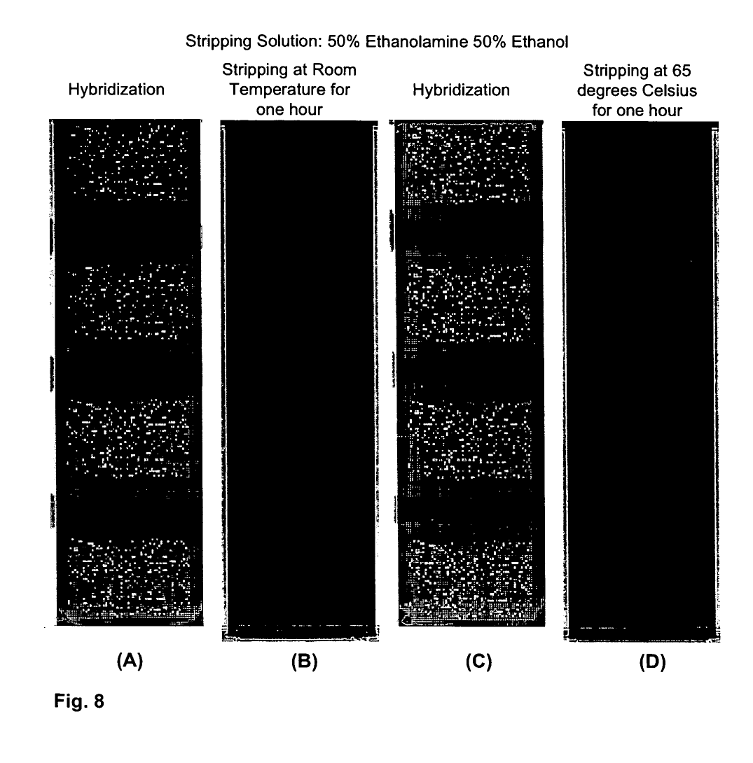

A microarray was prepared according to the procedures of Example 1. The stripping solution for removal of the labeled RNA comprised 50% ethanolamine and 50% ethanol. FIG. 8 provides images of microarrays before and after stripping at different temperatures and for a one hour stripping time. Three temperatures (22° C., 37° C. and 65° C.) were used; however, only the images of microarrays at the high and low temperatures are shown. The images are for the first stripping runs. The microarrays were actually rehybridized and stripped for five cycles. Room temperature stripping worked well; however when the individually spots were investigated under more intense laser light and higher magnification, a trace of material remained hybridized. Thus, stripping at 65° C. was better because essentially all hybridized target was removed, or at least material that could be detected under intense laser light at higher magnification.

### EXAMPLE 7

### Ethanolamine and Ethanol Stripping Time

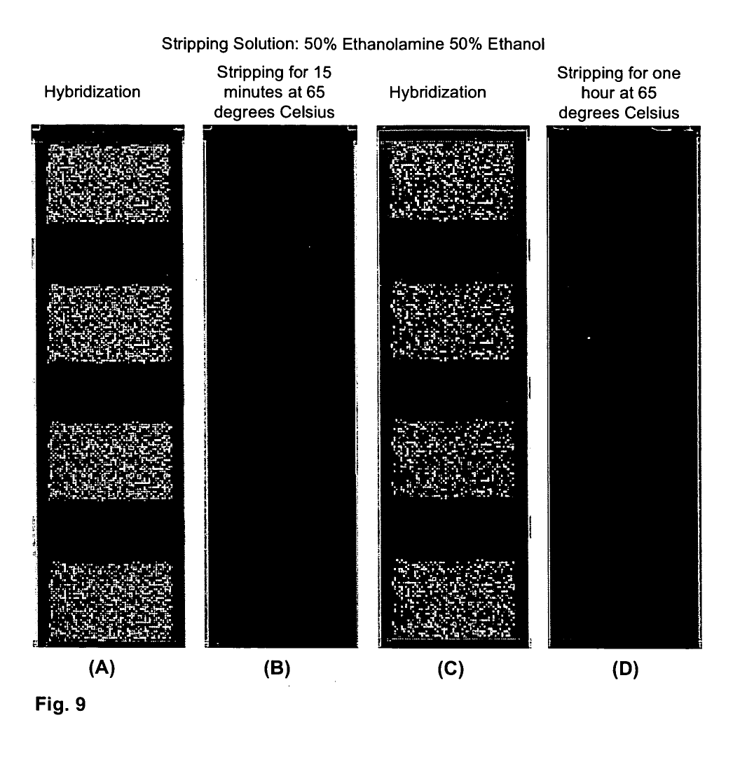

A microarray was prepared according to the procedures of Example 1. The stripping solution for removal of the labeled RNA comprised 50% ethanolamine and 50% ethanol. FIG. 9 provides images of microarrays before and after stripping at different times and at 65° C. Stripping time for the images shown was 15 minutes and one hour. The image for one hour stripping time produced less background and thus more hybridized target was removed. Thus, a one-hour stripping time was better.

### EXAMPLE 8

### Sodium Hydroxide Stripping Comparison

Three microarrays were prepared according to the procedures of Example 1. For each microarray, a stripping clamp with screw plugs was assembled to the respective microarrays. To two microarrays the following procedure was performed. For each microarray, 500 microliters of 0.5M NaOH was added into the stripping cap chamber using a pipette. This solution was removed with a pipette within about 1 minute of being added to each chamber. Next, another 500 microliters of fresh 0.5M NaOH was added to each chamber using a pipette. The two plugs into the solution portals were closed. The assembled stripping clamps with each microarray were incubated at ambient temperature (18-23° C.) for 15 minutes. After the incubation, the sodium hydroxide solution was removed with a pipette.

The stripping cap chamber was then filled with 500 microliters of a formulation of 50% ethanol and 50% ethanolamine. This formulation was removed within 1 minute using a pipette. A fresh aliquot of 500 microliters of the same formulations was added to each chamber. The solution portals were closed. The assembled stripping clamp with the microarray was incubated at 65° C. for 60 minutes. The solution was removed. The assembly was allowed to cool. Each chamber was rinsed with 95% ethanol, with nuclease-free water, and finally with 95% ethanol. Each microarray was removed from the stripping clamp and placed in a solution of 1×PBS and incubated for 20 minutes at 65° C. Each microarray was removed and covered with imaging solution for fluorescent imaging. Imaging was performed using an Axon Scanner.

To the third microarray, there was no exposure of the microarray to the sodium hydroxide solution. Instead, the stripping cap chamber was filled with 500 microliters of a formulation of 50% ethanol and 50% ethanolamine. This formulation was removed within 1 minute using a pipette. A fresh aliquot of 500 microliters of the same formulations was added to the chamber. The solution portals were closed. The assembled stripping clamp with the microarray was incubated at 65° C. for 60 minutes. The solution was removed. The assembly was allowed to cool. The chamber was rinsed with 95% ethanol, with nuclease-free water, and finally with 95% ethanol. The microarray was removed from the stripping clamp and placed in a solution of 1×PBS and incubated for 20 minutes at 65° C. The microarray was removed and covered with imaging solution for fluorescent imaging. Imaging was performed using an Axon Scanner.

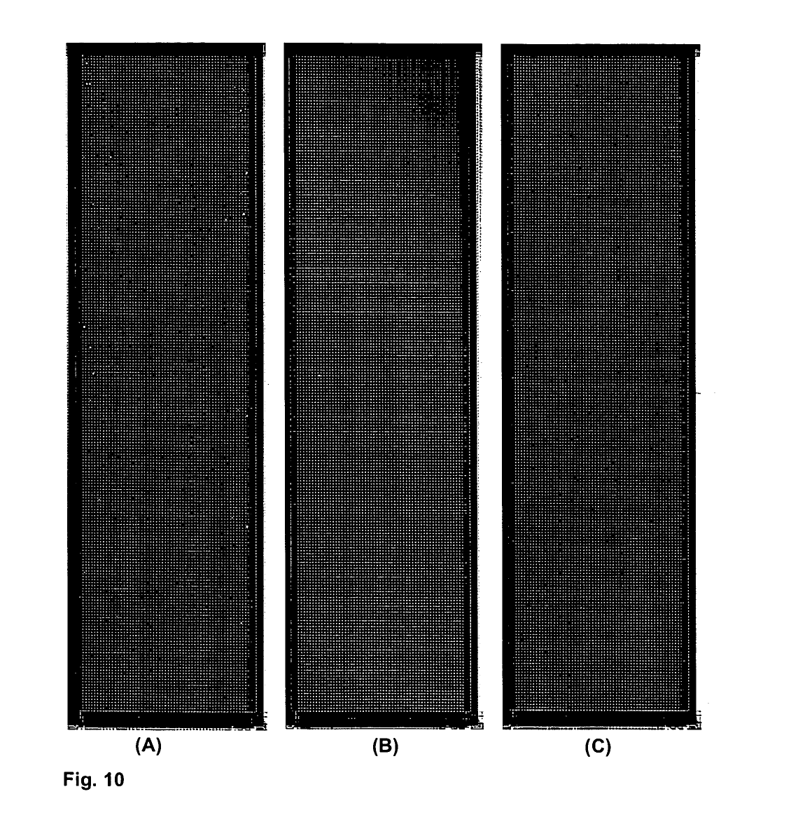

The results of imaging are shown in FIG. 10. FIG. 10 (A) shows the microarray that was not exposed to sodium hydroxide. FIGS. 10(B) and (C) shows the microarrays that were exposed to the sodium hydroxide solution. As can be seen in FIG. 10, the microarray without exposure to the sodium hydroxide solution shows incomplete stripping of target as evidenced by fluorescent spots on some of the electrodes of the microarray. The microarrays having exposure to the sodium hydroxide solution show complete stripping of the target as evidence by the lack of fluorescent spots at the electrodes.

What is claimed is:

1. A process for stripping a microarray for reuse, comprising:
(a) providing a microarray having probe oligonucleotides attached thereto and target oligonucleotides hybridized to the probe oligonucleotides; and
(b) incubating the microarray with a formulation comprising an organic solvent and an organic base, whereby the target oligonucleotides are substantially removed from the microarray.

2. The process of claim 1, wherein the temperature of incubating is from about room temperature to about 75 degrees Celsius.

3. The process of claim 1, wherein the time of incubating is from about 1 minute to about 24 hours.

4. The process of claim 1, wherein the time of incubating is about one hour and the temperature of incubating is about 65 degrees Celsius.

5. The process of claim 1, wherein the organic solvent concentration is about 50 percent by volume.

6. The process of claim 1, wherein the organic solvent is selected from the group consisting of ethanol, isopropanol, 1,1,1-trichloroethane, 1,1,2-trichloro-1,2,2-trifluoroethane, 1,1,2-trichloroethane, 1,4-dichlorobenzene, 1-butanol, 2-butanol, isobutanol, tert-butanol, 1-hexene, 1-propanol, 2-(2-butoxyethoxy)ethyl acetate, 2-butoxyethanol acetate, 2-butoxyethyl acetate, 2-ethoxyethanol acetate, 2-ethoxyethanol, 2-methoxyethanol acetate, 2-methoxyethanol, 2-methylhexane, 2-nitropropane, acetic acid, acetone alcohol, acetone, acetonitrile, allyl alcohol, benzene, benzotrifluoride, benzyl chloride, biphenyl, carbon disulfide, carbon tetrachloride, chlorobenzene, chlorobromomethane, cyclodecane, cycloheptane, cyclohexane, cyclohexanol, cyclohexanone, cyclononane, cyclooctane, cyclopentane, diacetone alcohol, dibromomethane, dichlorodiphenyltrichloroethane, dichloroethene, diemthyl sulfoxide, diethanolamine, diethyl ether, diethylene glycol, dimethyl ethanolamine, dimethyl formamide, dipropylene glycol, ethanol, ethyl acetate, ethyl benzene, ethyl ether, ethyl glycol acetate, ethyl glycol, ethylbenzene, ethylene glycol, formamide, formic acid, furfural, furfuryl alcohol, heptafluorocyclopentane, heptafluoropropyl methyl ether, heptane, hexachlorocyclohexane, hexane, isoamyl alcohol, isobutyl acetate, isobutyl alcohol, isobutyl isobutyrate, isomethoxynonafluorobutane, iso-methoxynonafluorobutane, isophorone, isopropyl acetate, iso-propyl alcohol, isopropylamine-striazine, methanol, methoxy propyl acetate, methyl amyl ketone, methyl chloride, methyl chloroform, methyl ethyl ketone, methyl glycol acetate methyl isobutyl ketone, methyl propyl ketone, methylene chloride, monochlorotoluene, monothiophosphate, n-amyl alcohol, n-butyl acetate, n-butyl alcohol, n-decane, nitrobenzene, nitromethane, n-methoxynonafluorobutane, n-methylpyrrolidone, n-nonane, n-octane, n-octyl alcohol, n-butyl acetate, n-methoxynonafluorobutane, n-pentane, n-propyl acetate, n-propyl alcohol, ortho-dichlorobenzene, perchloroethene, perchloroethylene, propylene glycol diacetate, propylene glycol, pyridine, t-amyl alcohol, t-butyl alcohol, tetrachloroethylene, tetrahydrofuran, toluene, trans-1,2-dichloroethylene, trichloroethene, trichloroethylene, trichlorofluoromethane, triethanolamine, triethylene gycol, vinyl choloride, xylene, and combinations thereof.

7. The process of claim 1, wherein the organic base is selected from the group consisting of ethanol amine, ethyl enediamine, adenine, guanine, cytocine, thymine, uracil, methylamine, ethyleneimine, dimethylamine, ethylamine, cysteamine, 1,2-ethanediamine, azetidine, propylamine, trimethylamine, 1-amino-2-methoxoythane, 1,2-propanediamine, 1,3-propanediamine, 1,2,3-triaminopropane, allantoin, pyrrolidine, morpholine, N,N-dimethylglycine, piperazine, butylamine, sec-butylamine, tert-butylamine, diethylamine, 1,4-butanediamine, 1,2-dimethylaminoethane, 4-pyridinamine, N-methylpyrrolidine, piperidine, 1-amino-2,2-dimethylpropane, diethylmethylamine, 3-methyl-1-butanamine, 2-methyl-1-butanamine, 3-pentanamine, pentylamine, cadaverine, cyclohexylamine, 1,2-dimethylpyrrolidine, 1-methylpiperidine, 3-amino-3-methylpentane, diisopropylamine, hexylamine, triethylamine, hexamethylenediamine, benzylamine, 1,2-dimethylpiperidine, 1-ethylpiperidine, 2-heptanamine, heptylamine, 2,2,4-trimethylpiperidine, dibutylamine, N-methyl-2-heptanamine, octylamine, 1-butylpiperidine, 2,2,6,6-tetramethylpiperidine, nonylamine, tryptamine, ephedrine, bornylamine, neobornylamine, butylcyclohexylamine, decylamine, undecylamine, dodecylamine, tridecylamine, tetradecylamine, pentadecyalamine, hexadecylamine, octadecylamine, and combinations thereof.

8. The process of claim 1, wherein the step of incubating the microarray with a formulation comprising an organic solvent and an organic base, further comprises:
(b₁) contacting the microarray to an aqueous base solution.

9. The process of claim 8, wherein the aqueous base solution is at a concentration of about 0.01 molar to about 5 molar.

10. The process of claim 8, wherein the aqueous base solution has a base selected from the group consisting of sodium hydroxide, potassium hydroxide, and ammonium hydroxide and combinations thereof.

11. The process of claim 8, wherein the temperature of contacting is about 2 to 95 degrees Celsius.

12. The process of claim 8, wherein the time of contacting is about 1 minute to about 60 minutes.

13. A process of stripping a microarray for reuse, comprising:
(a) providing a microarray having probe oligonucleotides attached thereto and target oligonucleotides hybridized to the probe oligonucleotides;
(b) contacting the microarray to an aqueous base solution; and
(c) incubating the microarray with a formulation comprising an organic solvent and an organic base, whereby the target oligonucleotides are substantially removed from the microarray.

14. The process of claim 13, wherein the aqueous base solution is at a concentration of about 0.01 molar to about 5 molar.

15. The process of claim 13, wherein the aqueous base solution has a base selected from the group consisting of sodium hydroxide, potassium hydroxide, and ammonium hydroxide and combinations thereof.

16. The process of claim 13, wherein the temperature of contacting is about 2 to 95 degrees Celsius.

17. The process of claim 13, wherein the time of contacting is about 1 minute to about 60 minutes.

18. The process of claim 13, wherein the temperature of incubating is from about room temperature to about 75 degrees Celsius.

19. The process of claim 13, wherein the time of incubating is from about 1 minute to about 24 hours.

20. The process of claim 13, wherein the organic solvent concentration is about 1 to 99 percent by volume.

21. The process of claim 13, wherein the organic solvent is selected from the group consisting of ethanol, isopropanol, 1,1,1-trichloroethane, 1,1,2-trichloro-1,2,2-trifluoroethane, 1,1,2-trichloroethane, 1,4-dichlorobenzene, 1-butanol, 2-butanol, isobutanol, tert-butanol, 1-hexene, 1-propanol, 2-(2-butoxyethoxy)ethyl acetate, 2-butoxyethanol acetate, 2-butoxyethyl acetate, 2-ethoxyethanol acetate, 2-ethoxyethanol, 2-methoxyethanol acetate, 2-methoxyethanol, 2-methylhexane, 2-nitropropane, acetic acid, acetone alcohol, acetone, acetonitrile, allyl alcohol, benzene, benzotrifluoride, benzyl chloride, biphenyl, carbon disulfide, carbon tetrachloride, chlorobenzene, chlorobromomethane, cyclodecane, cycloheptane, cyclohexane, cyclohexanol, cyclohexanone, cyclononane, cyclooctane, cyclopentane, diacetone alcohol, dibromomethane, dichlorodiphenyltrichloroethane, dichloroethene, diemthyl sulfoxide, diethanolamine, diethyl ether, diethylene glycol, dimethyl ethanolamine, dimethyl formamide, dipropylene glycol, ethanol, ethyl acetate, ethyl benzene, ethyl ether, ethyl glycol acetate, ethyl glycol, ethylbenzene, ethylene glycol, formamide, formic acid, furfural, furfuryl alcohol, heptafluorocyclopentane, heptafluoropropyl methyl ether, heptane, hexachlorocyclohexane, hexane, isoamyl alcohol, isobutyl acetate, isobutyl alcohol, isobutyl isobutyrate, isomethoxynonafluorobutane, iso-methoxynonafluorobutane, isophorone, isopropyl acetate, iso-propyl alcohol, isopropylamine-striazine, methanol, methoxy propyl acetate, methyl amyl ketone, methyl chloride, methyl chloroform, methyl ethyl ketone, methyl glycol acetate methyl isobutyl ketone, methyl propyl ketone, methylene chloride, monochlorotoluene, monothiophosphate, n-amyl alcohol, n-butyl acetate, n-butyl alcohol, n-decane, nitrobenzene, nitromethane, n-methoxynonafluorobutane, n-methylpyrrolidone, n-nonane, n-octane, n-octyl alcohol, n-butyl acetate, n-methoxynonafluorobutane, n-pentane, n-propyl acetate, n-propyl alcohol, ortho-dichlorobenzene, perchloroethene, perchloroethylene, propylene glycol diacetate, propylene glycol, pyridine, t-amyl alcohol, t-butyl alcohol, tetrachloroethylene, tetrahydrofuran, toluene, trans-1,2-dichloroethylene, trichloroethene, trichloroethylene, trichlorofluoromethane, triethanolamine, triethylene gycol, vinyl choloride, and xylene, and combinations thereof.

22. The process of claim 13, wherein the organic base is selected from the group consisting of ethanolamine, ethylenediamine, adenine, guanine, cytocine, thymine, uracil, methylamine, ethyleneimine, dimethylamine, ethylamine, cysteamine, 1,2-ethanediamine, azetidine, propylamine, trimethylamine, 1-amino-2-methoxoythane, 1,2-propanediamine, 1,3-propanediamine, 1,2,3-triaminopropane, allantoin, pyrrolidine, morpholine, N,N-dimethylglycine, piperazine, butylamine, sec-butylamine, tert-butylamine, diethylamine, 1,4-butanediamine, 1,2-dimethylaminoethane, 4-pyridinamine, N-methylpyrrolidine, piperidine, 1-amino-2,2-dimethylpropane, diethylmethylamine, 3-methyl-1-butanamine, 2-methyl-1-butanamine, 3-pentanamine, pentylamine, cadaverine, cyclohexylamine, 1,2-dimethylpyrrolidine, 1-methylpiperidine, 3-amino-3-methylpentane, diisopropylamine, hexylamine, triethylamine, hexamethylenediamine, benzylamine, 1,2-dimethylpiperidine, 1-ethylpiperidine, 2-heptanamine, heptylamine, 2,2,4-trimethylpiperidine, dibutylamine, N-methyl-2-heptanamine, octylamine, 1-butylpiperidine, 2,2,6,6-tetramethylpiperidine, nonylamine, tryptamine, ephedrine, bornylamine, neobornylamine, butylcyclohexylamine, decylamine, undecylamine, dodecylamine, tridecylamine, tetradecylamine, pentadecyalamine, hexadecylamine, and octadecylamine and combinations thereof.

23. A method of reusing a microarray comprising:
(a) providing a microarray having probe oligonucleotides attached thereto and target oligonucleotides hybridized to the probe oligonucleotides;
(b) stripping the target oligonucleotides from the microarray;
(c) rehybridizing a new set of target oligonucleotides to the probe oligonucleotides; and
(d) repeating steps (a) through (c) for each subsequent reuse of the microarray.

24. The method of claim 23, wherein the step of stripping the target oligonucleotides from the microarray, further comprises:
(b₁) incubating the microarray with a formulation comprising an organic solvent and an organic base.

25. The method of claim 24, wherein the step of incubating the microarray with a formulation comprising an organic solvent and an organic base, further comprises:
(b₁₁) contacting the microarray to an aqueous base solution.

26. The method of claim 23, wherein the step of stripping the target oligonucleotides from the microarray, further comprises:
(b₁) contacting the microarray to an aqueous base solution.
(b₂) incubating the microarray with a formulation comprising an organic solvent and an organic base.

27. A formulation for use in stripping target oligonucleotides from a microarray, comprising: an organic solvent and an organic base selected from the group consisting of ethanolamine and ethylenediamine.

28. The formulation of claim 27, wherein the organic solvent is selected from the group consisting of ethanol, isopropanol, 1,1,1-trichloroethane, 1,1,2-trichloro-1,2,2-trifluoroethane, 1,1,2-trichloroethane, 1,4-dichlorobenzene, 1-butanol, 2-butanol, isobutanol, tert-butanol, 1-hexene, 1-propanol, 2-(2-butoxyethoxy)ethyl acetate, 2-butoxyethanol acetate, 2-butoxyethyl acetate, 2-ethoxyethanol acetate, 2-ethoxyethanol, 2-methoxyethanol acetate, 2-methoxyethanol, 2-methylhexane, 2-nitropropane, acetic acid, acetone alcohol, acetone, acetonitrile, allyl alcohol, benzene, benzotrifluoride, benzyl chloride, biphenyl, carbon disulfide, carbon tetrachloride, chlorobenzene, chlorobromomethane, cyclodecane, cycloheptane, cyclohexane, cyclohexanol, cyclohexanone, cyclononane, cyclooctane, cyclopentane, diacetone alcohol, dibromomethane, dichlorodiphenyltrichloroethane, dichloroethene, diemthyl sulfoxide, diethanolamine, diethyl ether, diethylene glycol, dimethyl ethanolamine, dimethyl formamide, dipropylene glycol, ethanol, ethyl acetate, ethyl benzene, ethyl ether, ethyl glycol acetate, ethyl glycol, ethylbenzene, ethylene glycol, formamide, formic acid, furfural, furfuryl alcohol, heptafluorocyclopentane, heptafluoropropyl methyl ether, heptane, hexachlorocyclohexane, hexane, isoamyl alcohol, isobutyl acetate, isobutyl alcohol, isobutyl isobutyrate, isomethoxynonafluorobutane, iso-methoxynonafluorobutane, isophorone, isopropyl acetate, iso-propyl alcohol, isopropylamine-striazine, methanol, methoxy propyl acetate, methyl amyl ketone, methyl chloride, methyl chloroform, methyl ethyl ketone, methyl glycol acetate methyl isobutyl ketone, methyl propyl ketone, methylene chloride, monochlorotoluene, monothiophosphate, n-amyl alcohol, n-butyl acetate, n-butyl alcohol, n-decane, nitrobenzene, nitromethane, n-methoxynonafluorobutane, n-methylpyrrolidone, n-nonane, n-octane, n-octyl alcohol, n-butyl acetate, n-methoxynonafluorobutane, n-pentane, n-propyl acetate, n-propyl alcohol, ortho-dichlorobenzene, perchloroethene, perchloroethylene, propylene glycol diacetate, propylene glycol, pyridine, t-amyl alcohol, t-butyl alcohol, tetrachloroethylene, tetrahydrofuran, toluene, trans-1,2-dichloroethylene, trichloroethene, trichloroethylene, trichlorofluoromethane, triethanolamine, triethylene gycol, vinyl choloride, xylene, and combinations thereof.
